(19) United States
(12) Patent Application Publication
(10) Pub. No.: US 2021/0171422 A1
(43) Pub. Date: Jun. 10, 2021

(54) Liquid Phase Transalkylation Process

(71) Applicant: ExxonMobil Chemical Patents Inc., Baytown,, TX (US)

(72) Inventors: Jane C. Cheng, Bethlehem, PA (US); Darryl D Lacy, Easton, PA (US); Scott J Seigel, Allentown, PA (US); Brandon J O'Neil, Lebanon, NJ (US); Tan-Jen Chen, Seattle, WA (US); Todd E Deetjen, Houston, TX (US)

(21) Appl. No.: 16/071,398

(22) PCT Filed: Feb. 10, 2017
(86) PCT No.: PCT/US2017/017299

§ 371 Date: Jul. 19, 2018

Related U.S. Application Data
(60) Provisional application No. 62/313,966, filed on Mar. 28, 2016.

(30) Foreign Application Priority Data
May 19, 2016 (EP) 16170265.9

(51) Int. Cl. C07C 6/12; B01J 29/74

(52) U.S. Cl. CPC C07C 6/126; C07C 2529/068; B01J 29/7476

(57) ABSTRACT

Methods and corresponding catalysts are provided for transalkylation of 1-ring (C₉₊) aromatic compounds, such as transalkylation to form para-xylene and/or other xylenes. Suitable catalysts include molecular sieves having a 3-D 12-member ring framework structure, molecular sieves having a 1-D 12-member ring framework structure, acidic microporous materials with a pore channel size of at least 6.0 Angstroms, and/or molecular sieves having a MWW framework structure. The methods include performing transalkylation where at least a portion of the feed to the transalkylation process is in the liquid phase. Optionally, the transalkylation conditions can correspond to conditions where a continuous liquid phase is present within the reaction environment. Some embodiments include liquid phase transalkylation processes for naphthalene-containing feedstock streams.

### PRIORITY

This application claims priority to and the benefit of U.S. Provisional Application No. 62/313,966, filed Mar. 28, 2016, and EP Application No. 16170265.9, filed May 19, 2016, the entire contents of each being incorporated herein by reference for all purposes.

### TECHNICAL FIELD

Systems and methods are provided for transalkylation of aromatic compounds, such as transalkylation between mononuclear aromatic compounds.

### BACKGROUND

Production of xylenes from mixed aromatic streams is an important process commercially. Due to various equilibria, aromatic formation processes tend to produce relatively low amounts of xylenes relative to other single ring aromatics. One option for converting a mixed feed of aromatics to produce additional xylenes is to perform a transalkylation process. Conventional transalkylation processes are typically performed under gas phase conditions, including temperatures of at least about 380° C.

U.S. Pat. No. 7,553,791 teaches a process for the conversion of a feedstock containing C₉₊ aromatic hydrocarbons to produce a resulting product containing lighter aromatic products and less than about 0.5 wt % of ethylbenzene based on the weight of C₈ aromatics fraction of said resulting product, said process comprising contacting said feedstock under transalkylation reaction conditions with a catalyst composition comprising: (i) an acidity component having an alpha value of at least 300; and (ii) a hydrogenation component having hydrogenation activity (ratio of ethylene to ethane in transalkylation product under defined conditions) of at least 300, the C₉₊ aromatic hydrocarbons being converted under said transalkylation reaction conditions to a reaction product containing xylenes. Preferably, the aromatic product contains less than about 0.3 wt % of ethylbenzene based on the weight of C₈ aromatics fraction of said resulting product. More preferably, the aromatic product contains less than about 0.2 wt % of ethylbenzene based on the weight of C₈ aromatics fraction of said resulting product. Preferably, the acidity component comprises a molecular sieve selected from the group consisting of one or more of a first molecular sieve having a MTW structure, a molecular sieve having a MOR structure, and a porous crystalline inorganic oxide material having an X-ray diffraction pattern including d-spacing maxima (Å) at 12.4±0.25, 6.9±0.15, 3.57±0.07 and 3.42±0.07. More preferably, the catalyst comprises a molecular sieve ZSM-12. Alternatively, the porous crystalline inorganic oxide material is selected from the group consisting of one or more of MCM-22, PSH-3, SSZ-25, MCM-36, MCM-49 and MCM-56. In another embodiment, the catalyst comprises second molecular sieve having a constraint index ranging from 3 to 12. Preferably, the second molecular sieve is ZSM-5. Preferably, the catalyst comprises two molecular sieves, the first molecular sieve is ZSM-12, and the second molecular sieve is ZSM-5. Conveniently, the catalyst composition is particulate and the first and second molecular sieves are each contained in the same catalyst particles. Preferably, the hydrogenation component is selected from the group consisting of one or more of a Group VIIIB and Group VIIB metal. More preferably, the hydrogenation component is selected from the group consisting of one or more of rhenium, platinum, and palladium.

A catalyst system for the transalkylation of C₉₊ aromatics with C₆-C₇ aromatics is disclosed in U.S. Pat. No. 7,663,010. The catalyst system described therein comprises (a) a first catalyst comprising a molecular sieve having a Constraint Index in the range of 3-12 (e.g., a 10 MR molecular sieve, such as ZSM-5, ZSM-11, ZSM-22, and ZSM-23) and a metal catalyzing the saturation of the olefins formed by the dealkylation reactions; and (b) a second catalyst comprising a molecular sieve having a Constraint Index in the range of less than 3 (e.g., a 12 MR molecular sieve, such as ZSM-12, MOR, zeolite beta, MCM-22 family molecular sieve) and optionally a metal which may be the same or different to the metal on the first catalyst. U.S. Pat. Nos. 8,163,966 and 9,034,780 describe additional catalyst systems and methods for performing transalkylation on mixed aromatic feeds.

Conventionally, transalkylation processes have typically been performed under gas phase transalkylation conditions. However, performing transalkylation on a feed that is at least partially in the liquid phase would be desirable to minimize energy consumption.

### BRIEF SUMMARY

At least some embodiments disclosed herein are directed to performing transalkylation under at least partially liquid phase conditions. Performing transalkylation on a feed that is at least partially in the liquid phase allows for an improved catalyst lifetime and/or an improved selectivity for production of aromatics having a desired number of carbons (such as C₈ aromatics) at lower severity reaction conditions. Other potential benefits of performing transalkylation under liquid phase conditions can include one or more of lower reaction temperature, reduced or minimized energy consumption, reduced or minimized by-product formation, and/or improved yield of C₈ aromatics.

In an aspect, a method for liquid phase transalkylation of aromatic compounds is provided. The method includes exposing an aromatic feedstock comprising C₉₊ aromatics and at least one of benzene and toluene to a transalkylation catalyst under effective transalkylation conditions to form a transalkylation effluent. The mole fraction of aromatic compounds in the liquid phase in the feedstock, relative to the total amount of aromatic compounds in the feedstock, of at least about 0.01 under the effective transalkylation conditions. The presence of a liquid mole fraction in the transalkylation reaction environment allows for improved and/or modified transalkylation activity relative to gas phase transalkylation conditions. The resulting transalkylation effluent has a higher weight percentage of C₈ aromatics than the feedstock.

The transalkylation catalyst comprises at least one of the following: a molecular sieve with a 3-dimensional 12-member ring or larger pore network; a molecular sieve with a 1-dimensional 12-member ring or larger pore network, wherein the 1-dimensional channel has a pore channel size of at least 6.0 Angstroms; an acidic microporous material with a pore channel size of at least 6.0 Angstroms; and a molecular sieve having a MWW framework. Optionally, the catalyst can further comprise 0.01 wt % to 5.0 wt % of a Group 6-11 metal, such as Pd, Pt, Ni, Rh, Cu, Sn, or a combination thereof. In aspects where the catalyst includes a molecular sieve with an MWW framework, the catalyst can optionally comprise 0.01 wt % to 5.0 wt % of a Group 8-10 noble metal, such as Pd. Optionally, the Group 6-11 metal or the Group 8-10 metal can correspond to a bimetallic metal.

### BRIEF DESCRIPTION OF THE FIGURES

FIG. 1 shows examples of the mole fraction of a feed in the liquid phase at various temperature and pressure conditions.

FIG. 2 shows feed conversion and xylene yields for transalkylation with a mordenite catalyst.

FIG. 3 shows feed conversion and xylene yields for transalkylation with a MCM-22 catalyst.

FIG. 4 shows feed conversion and xylene yields for transalkylation with a 0.15 wt % Pd/FAU catalyst.

FIG. 5 shows feed conversion and xylene yields for transalkylation with a MCM-49 catalyst.

FIG. 6 shows xylene yields for transalkylation with a MCM-49 catalyst.

FIG. 7 shows xylene yields for transalkylation with a 0.15 wt % Pd/MCM-49 catalyst.

FIG. 8 shows xylene yields for transalkylation with a zeolite Beta catalyst.

FIG. 9 shows xylene yields for transalkylation with a 0.15 wt % Pd/zeolite Beta catalyst.

FIG. 10 shows xylene yields for transalkylation with a high Si/Al₂ ratio zeolite Beta catalyst.

FIG. 11 shows xylene yields for transalkylation with a FAU catalyst.

FIG. 12 shows xylene yields from transalkylation with the various catalysts used in FIGS. 6-11.

FIG. 13 shows relative xylene yields and benzene yields from transalkylation with the various catalysts used in FIGS. 6-11.

FIG. 14 shows production of heavy aromatic compounds (C₁₀₊) from transalkylation with the various catalysts used in FIGS. 6-11.

FIG. 15 shows results from transalkylation of naphthalene in the presence of MCM-22 with various feedstocks.

FIG. 16 shows additional xylene yield results for MCM-49 and 0.15 wt % Pd/MCM-49.

FIG. 17 shows naphthalene conversion over time during transalkylation of a naphthalene-containing feedstock.

### DETAILED DESCRIPTION OF THE EMBODIMENTS

### Overview and Definitions

In various aspects, methods and corresponding catalysts are provided for transalkylation of 1-ring (C₉₊) aromatic compounds to form para-xylene and/or other xylenes. The methods include performing transalkylation where at least a portion of the feed to the transalkylation process is in the liquid phase. Optionally, the transalkylation conditions can correspond to conditions where a continuous liquid phase is present within the reaction environment. Conventionally, transalkylation processes have typically been performed under gas phase transalkylation conditions. However, a variety of benefits can be obtained by performing transalkylation under liquid phase conditions. For example, performing transalkylation on a feed that is at least partially in the liquid phase allows for an improved catalyst lifetime and/or an improved selectivity for production of aromatics having a desired number of carbons (such as C₈ aromatics) at lower severity reaction conditions. Other potential benefits of performing transalkylation under liquid phase conditions include one or more of lower reaction temperature, reduced or minimized energy consumption, reduced or minimized by-product formation, and/or improved yield of C₈ aromatics.

Aromatics formation processes often produce a variety of single-ring aromatics that include C₆, C₇, C₈, C₉, and C₁₀ aromatics. When a desired product is, for example, a C₈ aromatic, methylation processes can be used to convert C₆ and/or C₇ aromatics into C₈ aromatics. It would be desirable to also convert higher carbon content aromatics, such as C₉ and/or C₁₀ aromatics in this example, into C₈ aromatics. Transalkylation processes can allow for this type of modification (removal, addition, and/or replacement) of the alkyl side chains of an aromatic compound.

In some aspects, a suitable catalyst for performing liquid phase transalkylation comprises a molecular sieve with a 3-dimensional 12-member ring (or larger) pore network. Without being bound by any particular theory, a molecular sieve with a 3-dimensional 12-member ring pore network can provide unexpected activity for transalkylation. Optionally, the Si/Al₂ ratio of the molecular sieve can also be selected to facilitate improved yield of C₈ aromatics at a given temperature. Optionally, the catalyst comprising a 3-D 12-member ring molecular sieve can further include a hydrogenation metal supported on the catalyst.

In other aspects, a suitable catalyst for performing liquid phase transalkylation comprises a molecular sieve with a 1-dimensional 12-member ring (or larger) pore network, where the 1-dimensional channel has a pore channel size of at least 6.0 Angstroms, or at least 6.3 Angstroms. A pore channel with a larger pore channel size can also allow for improved activity for transalkylation. For example, the MOR framework structure has a pore 12-member ring pore channel size of about 6.45 Angstroms, while the MEI framework structure (ZSM-18) has a pore channel size of about 6.9 Angstroms. By contrast, the MTW framework structure (ZSM-12) has a pore channel size of about 5.7 Angstroms. Optionally, the Si/Al₂ ratio of the molecular sieve can also be selected to facilitate improved yield of C₈ aromatics at a given temperature. Optionally, the catalyst comprising a 1-D 12-member ring molecular sieve can further include a hydrogenation metal supported on the catalyst.

In still other aspects, a suitable catalyst for performing liquid phase transalkylation comprises an acidic microporous material that has a largest pore channel corresponding to a 12-member ring or larger, and/or that has a pore channel size of at least 6.0 Angstroms, or at least 6.3 Angstroms and/or that has another active surface having a size of at least 6.0 Angstroms. Optionally, the catalyst comprising a microporous material can further include a hydrogenation metal supported on the catalyst. Optionally, the microporous material can be a zeolite or another type of molecular sieve.

In yet other aspects, a suitable catalyst for performing liquid phase transalkylation comprises a molecular sieve having a MWW framework. It is noted that although the MWW framework has 10-member ring channels as the largest traditional pore channel, an MWW framework crystal can also have surface locations that provide a structure similar to a 12-member ring pore. Without being bound by any particular theory, it is believed that these surface locations allow an MWW framework molecular sieve to serve as a transalkylation catalyst. Optionally, a catalyst including an MWW framework molecular sieve can further include a hydrogenation metal supported on the catalyst.

In this discussion, performing a liquid phase transalkylation reaction can correspond to performing transalkylation under reaction conditions where at least a portion of the aromatic compounds in the reaction environment are in the liquid phase. The mole fraction of aromatic compounds in the liquid phase, relative to the total aromatics, hereinafter termed “liquid mole fraction,” can be at least 0.01, or at least 0.05, or at least 0.08, or at least 0.1, or at least 0.15, or at least 0.2, or at least 0.3, or at least 0.4, or at least 0.5, and optionally up to having substantially all aromatic compounds in the liquid phase.

Due to the difference in volume between gases and liquids, the volume fraction of liquid phase in a reactor can be smaller than the mole fraction. As a rough approximation, the volume of a typical gas phase can be estimated using the ideal gas law. For transalkylation, the volume of a typical aromatic liquid can be estimated by assuming an average molecular weight of about 100-120 g/mol and a liquid phase density of about 0.8 g/ml-0.9 g/ml. Under these assumptions, at a temperature of about 300° C. and a partial pressure of aromatic compounds of about 300 psig, having a liquid mole fraction of about 0.5 would be expected to correspond to having a liquid volume fraction of about 5%-10% of the volume of the reaction environment. For a liquid mole fraction of about 0.1, the corresponding liquid volume would be expected to correspond to 0.5%-1.0% of the reaction environment. Without being bound by any particular theory, it is believed that formation of even small amounts of a condensed (liquid) phase can substantially alter the nature of a transalkylation reaction environment. Such a liquid phase can potentially form preferentially at surfaces within a reaction environment, such as at the surfaces of catalyst particles. Thus, small amounts of liquid formation can potentially be sufficient to effectively provide liquid phase reaction conditions.

In aspects where the mole fraction of aromatics in the liquid phase is at least 0.4, performing a liquid phase transalkylation corresponds to performing transalkylation under conditions where the liquid phase corresponds to at least about 5% of the total volume of the reaction environment. In such aspects, a continuous liquid phase may optionally be formed in the reaction environment, so that at least 30 vol % of the liquid in the reaction environment forms a single, continuous phase, or at least 50 vol %, or at least 70 vol %. This can be in contrast, for example, to performing transalkylation under trickle-bed conditions, where a plurality of separate liquid phases can form within a fixed catalyst bed. In other aspects, the transalkylation reaction can be performed under trickle-bed conditions.

As used in this specification, the term “framework type” is used in the sense described in the “Atlas of Zeolite Framework Types,” 2001.

The xylene yield, as used herein, is calculated by dividing the total weight of the xylene isomers (para-, meta-, and ortho-xylene) by the total weight of the product stream. The total weight of the xylene isomers can be calculated by multiplying the weight percentage of the xylene isomers, as determined by gas chromatography, by the total weight of the product stream.

Weight of molecular sieve, weight of binder, weight of catalyst composition, weight ratio of molecular sieve over catalyst composition, and weight ratio of binder over catalyst composition are calculated based on calcined weight (at 510° C. in air for 24 hours), i.e., the weight of the molecular sieve, the binder, and the catalyst composition are calculated based on the weight of the molecular sieve, the binder, and the catalyst composition after being calcined at 510° C. in air for twenty-four hours.

The term “aromatic” as used herein is to be understood in accordance with its art-recognized scope which includes alkyl substituted and unsubstituted mono- and polynuclear compounds.

The term “Cₙ” hydrocarbon wherein n is an positive integer, e.g., 1, 2, 3, 4, 5, 6, 7, 8, 9, 10, 11, 12, as used herein means a hydrocarbon having n number of carbon atom(s) per molecular. For example, Cₙ aromatics means an aromatic hydrocarbon having n number of carbon atom(s) per molecule. The term “Cₙ₊” hydrocarbon wherein n is an positive integer, e.g., 1, 2, 3, 4, 5, 6, 7, 8, 9, 10, 11, 12, as used herein means a hydrocarbon having at least n number of carbon atom(s) per molecule. The term “Cₙ₋” hydrocarbon wherein n is an positive integer, e.g., 1, 2, 3, 4, 5, 6, 7, 8, 9, 10, 11, 12, as used herein means a hydrocarbon having no more than n number of carbon atom(s) per molecule.

The term “Cₙ feedstock”, wherein n is a positive integer, e.g., 1, 2, 3, 4, 5, 6, 7, 8, 9, 10, 11, 12, as used herein, means that the Cₙ feedstock comprises greater than 50 wt % (or greater than 75 wt % or greater than 90 wt %) of hydrocarbons having n number of carbon atom(s) per molecule. The term “Cₙ₊ feedstock”, wherein n is a positive integer, e.g., 1, 2, 3, 4, 5, 6, 7, 8, 9, 10, 11, 12, as used herein, means that the Cₙ₊ feedstock comprises greater than 50 wt % (or greater than 75 wt % or greater than 90 wt %) of hydrocarbons having at least n number of carbon atom(s) per molecule. The term “Cₙ₋ feedstock” wherein n is a positive integer, e.g., 1, 2, 3, 4, 5, 6, 7, 8, 9, 10, 11, 12, as used herein, means that the Cₙ₋ feedstock comprises greater than 50 wt % (or greater than 75 wt % or greater than 90 wt %) of hydrocarbons having no more than n number of carbon atom(s) per molecule. The term “Cₙ aromatic feedstock”, wherein n is a positive integer, e.g., 1, 2, 3, 4, 5, 6, 7, 8, 9, 10, 11, 12, as used herein, means that the Cₙ aromatic feedstock comprises greater than 50 wt % (or greater than 75 wt % or greater than 90 wt %) of aromatic hydrocarbons having n number of carbon atom(s) per molecule. The term “Cₙ₊ aromatic feedstock”, wherein n is a positive integer, e.g., 1, 2, 3, 4, 5, 6, 7, 8, 9, 10, 11, 12, as used herein, means that the Cₙ₊ aromatic feedstock comprises greater than 50 wt % (or greater than 75 wt % or greater than 90 wt %) of aromatic hydrocarbons having at least n number of carbon atom(s) per molecule. The term “Cₙ₋ aromatic feedstock” wherein n is a positive integer, e.g., 1, 2, 3, 4, 5, 6, 7, 8, 9, 10, 11, 12, as used herein, means that the Cₙ₋ aromatic feedstock comprises greater than 50 wt % (or greater than 75 wt % or greater than 90 wt %) of aromatic hydrocarbons having no more than n number of carbon atom(s) per molecule.

### Liquid Phase Transalkylation of Aromatics

For liquid phase alkylation, in some aspects a suitable transalkylation catalyst includes a molecular sieve with a framework structure having a 3-dimensional network of 12-member ring pore channels. Examples of framework structures having a 3-dimensional 12-member ring are the framework structures corresponding to faujasite (such as zeolite X or Y, including USY), *BEA (such as zeolite Beta), BEC (polymorph C of Beta), CIT-1 (CON), MCM-68 (MSE), hexagonal faujasite (EMT), ITQ-7 (ISV), ITQ-24 (IWR), and ITQ-27 (IWV), preferably faujasite, hexagonal faujasite, and Beta (including all polymorphs of Beta). It is noted that the materials having a framework structure including a 3-dimensional network of 12-member ring pore channels can correspond to zeolites, silicoaluminophosphates, aluminophosphates, and/or any other convenient combination of framework atoms.

Additionally or alternately, a suitable transalkylation catalyst includes a molecular sieve with a framework structure having a 1-dimensional network of 12-member ring pore channels, where the pore channel has a pore channel size of at least 6.0 Angstroms, or at least 6.3 Angstroms. The pore channel size of a pore channel is defined herein to refer to the maximum size sphere that can diffuse along a channel Examples of framework structures having a 1-dimensional 12-member ring pore channel can include, but are not limited to, mordenite (MOR), zeolite L (LTL), and ZSM-18 (MEI). It is noted that the materials having a framework structure including a 1-dimensional network of 12-member ring pore channels can correspond to zeolites, silicoaluminophosphates, aluminophosphates, and/or any other convenient combination of framework atoms.

Additionally or alternately, a suitable transalkylation catalyst includes a molecular sieve having the MWW framework structure. Although the MWW framework structure does not have 12-member ring pore channels, the MWW framework structure does include surface sites that have features similar to a 12-member ring opening. Examples of molecular sieves having MWW framework structure include MCM-22, MCM-49, MCM-56, MCM-36, EMM-10, EMM-13, ITQ-1, ITQ-2, UZM-8, MIT-1, and interlayer expanded zeolites. It is noted that the materials having an MWW framework structure can correspond to zeolites, silicoaluminophosphates, aluminophosphates, and/or any other convenient combination of framework atoms.

Additionally or alternately, a suitable transalkylation catalyst includes an acidic microporous material that has a largest pore channel corresponding to a 12-member ring or larger, and/or that has a pore channel size of at least 6.0 Angstroms, or at least 6.3 Angstroms and/or that has another active surface having a size of at least 6.0 Angstroms. It is noted that such microporous materials can correspond to zeolites, silicoaluminophosphates, aluminophosphates, and/or materials that are different from molecular sieve type materials.

The molecular sieve can optionally be characterized based on having a composition with a molar ratio YO₂ over X₂O₃=n, wherein X is a trivalent element, such as aluminum, boron, iron, indium and/or gallium, preferably aluminum and/or gallium, and Y is a tetravalent element, such as silicon, tin and/or germanium, preferably silicon. For a MWW framework molecular sieve, n can be less than about 50, e.g., from about 2 to less than about 50, usually from about 10 to less than about 50, more usually from about 15 to about 40. For a molecular sieve having the framework structure of Beta and/or its polymorphs, n can be about 10 to about 60, or about 10 to about 50, or about 10 to about 40, or about 20 to about 60, or about 20 to about 50, or about 20 to about 40, or about 60 to about 250, or about 80 to about 250, or about 80 to about 220, or about 10 to about 400, or about 10 to about 250, or about 60 to about 400, or about 80 to about 400. For a molecular sieve having the framework structure FAU, n can be about 2 to about 400, or about 2 to about 100, or about 2 to about 80, or about 5 to about 400, or about 5 to about 100, or about 5 to about 80, or about 10 to about 400, or about 10 to about 100, or about 10 to about 80. Optionally, the above n values can correspond to n values for a ratio of silica to alumina in the MWW, *BEA, and/or FAU framework molecular sieve. In such optional aspects, the molecular sieve can optionally correspond to an aluminosilicate and/or a zeolite.

Optionally, the catalyst comprises 0.01 wt % to 5.0 wt %, or 0.01 wt % to 2.0 wt %, or 0.01 wt % to 1.0 wt %, or 0.05 wt % to 5.0 wt %, or 0.05 wt % to 2.0 wt %, or 0.05 wt % to 1.0 wt %, or 0.1 wt % to 5.0 wt %, or 0.1 to 2.0 wt %, or 0.1 wt % to 1.0 wt %, of a metal element of Groups 5-11 (according to the IUPAC Periodic Table). The metal element may be at least one hydrogenation component, such as one or more metals selected from Group 5-11 and 14 of the Periodic Table of the Elements, or a mixture of such metals, such as a bimetallic (or other multimetallic) hydrogenation component. Optionally, the metal can be selected from Groups 8-10, such as a Group 8-10 noble metal. Specific examples of useful metals are iron, tungsten, vanadium, molybdenum, rhenium, chromium, manganese, ruthenium, osmium, nickel, cobalt, rhodium, iridium, copper, tin, noble metals such as platinum or palladium, and combinations thereof. Specific examples of useful bimetallic combinations (or multimetallic combinations) are those where Pt is one of the metals, such as Pt/Sn, Pt/Pd, Pt/Cu, and Pt/Rh. In some aspects, the hydrogenation component is palladium, platinum, rhodium, copper, tin, or a combination thereof. The amount of the hydrogenation component can be selected according to a balance between hydrogenation activity and catalytic functionality. For a hydrogenation component including two or more metals (such as a bimetallic hydrogenation component), the ratio of a first metal to a second metal can range from 1:1 to about 1:100 or more, preferably 1:1 to 1:10.

Optionally, a suitable transalkylation catalyst can be a molecular sieve that has a constraint index of 1-12, optionally but preferably less than 3. The constraint index can be determined by the method described in U.S. Pat. No. 4,016,218, which is incorporated herein by reference with regard to the details of determining a constraint index.

Additionally or alternately, a transalkylation catalyst (such as a transalkylation catalyst system) can be used that has a reduced or minimized activity for dealkylation. The Alpha value of a catalyst can provide an indication of the activity of a catalyst for dealkylation. In various aspects, the transalkylation catalyst can have an Alpha value of about 100 or less, or about 50 or less, or about 20 or less, or about 10 or less, or about 1 or less. The alpha value test is a measure of the cracking activity of a catalyst and is described in U.S. Pat. No. 3,354,078 and in the Journal of Catalysis, Vol. 4, p. 527 (1965); Vol. 6, p. 278 (1966); and Vol. 61, p. 395 (1980), each incorporated herein by reference as to that description. The experimental conditions of the test used herein include a constant temperature of 538° C. and a variable flow rate as described in detail in the Journal of Catalysis, Vol. 61, p. 395.

It may be desirable to incorporate a molecular sieve in the catalyst composition with another material that is resistant to the temperatures and other conditions employed in the transalkylation process of the disclosure. Such materials include active and inactive materials and synthetic or naturally occurring zeolites, as well as inorganic materials such as clays, silica, hydrotalcites, perovskites, spinels, inverse spinels, mixed metal oxides, and/or metal oxides such as alumina, lanthanum oxide, cerium oxide, zirconium oxide, and titania. The inorganic material may be either naturally occurring, or in the form of gelatinous precipitates or gels including mixtures of silica and metal oxides.

Use of a material in conjunction with each molecular sieve, i.e., combined therewith or present during its synthesis, which itself is catalytically active, may change the conversion and/or selectivity of the catalyst composition. Inactive materials suitably serve as diluents to control the amount of conversion so that transalkylated products can be obtained in an economical and orderly manner without employing other means for controlling the rate of reaction. These catalytically active or inactive materials may be incorporated into, for example, alumina, to improve the crush strength of the catalyst composition under commercial operating conditions. It is desirable to provide a catalyst composition having good crush strength because in commercial use, it is desirable to prevent the catalyst composition from breaking down into powder-like materials.

Naturally occurring clays that can be composited with each molecular sieve as a binder for the catalyst composition include the montmorillonite and kaolin family, which families include the subbentonites, and the kaolins commonly known as Dixie, McNamee, Georgia and Florida clays or others in which the main mineral constituent is halloysite, kaolinite, dickite, nacrite or anauxite. Such clays can be used in the raw state as originally mined or initially subjected to calcination, acid treatment or chemical modification.

In addition to the foregoing materials, each molecular sieve (and/or other microporous material) can be composited with a binder or matrix material, such as an inorganic oxide selected from the group consisting of silica, alumina, zirconia, titania, thoria, beryllia, magnesia, lanthanum oxide, cerium oxide, manganese oxide, yttrium oxide, calcium oxide, hydrotalcites, perovskites, spinels, inverse spinels, and combinations thereof, such as silica-alumina, silica-magnesia, silica-zirconia, silica-thoria, silica-beryllia, silica-titania, as well as ternary compositions such as silica-alumina-thoria, silica-alumina-zirconia, silica-alumina-magnesia and silica-magnesia-zirconia. It may also be advantageous to provide at least a part of the foregoing porous matrix binder material in colloidal form so as to facilitate extrusion of the catalyst composition.

In some aspects, a molecular sieve (and/or other microporous material) can be used without an additional matrix or binder. In other aspects, a molecular sieve/microporous material can be admixed with a binder or matrix material so that the final catalyst composition contains the binder or matrix material in an amount ranging from 5 to 95 wt %, and typically from 10 to 60 wt %.

Prior to use, steam treatment of the catalyst composition may be employed to minimize the aromatic hydrogenation activity of the catalyst composition. In the steaming process, the catalyst composition is usually contacted with from 5 to 100% steam, at a temperature of at least 260° to 650° C. for at least one hour, specifically 1 to 20 hours, at a pressure of 100 to 2590 kPa-a.

A hydrogenation component can be incorporated into the catalyst composition by any convenient method. Such incorporation methods can include co-crystallization, exchange into the catalyst composition, liquid phase and/or vapor phase impregnation, or mixing with the molecular sieve and binder, and combinations thereof. For example, in the case of platinum, a platinum hydrogenation component can be incorporated into the catalyst by treating the molecular sieve with a solution containing a platinum metal-containing ion. Suitable platinum compounds for impregnating the catalyst with platinum include chloroplatinic acid, platinous chloride and various compounds containing the platinum ammine complex, such as Pt(NH₃)₄Cl₂.H₂O or (NH₃)₄Pt(NO₃)₂.H₂O. Palladium can be impregnated on a catalyst in a similar manner.

Alternatively, a compound of the hydrogenation component may be added to the molecular sieve when it is being composited with a binder, or after the molecular sieve and binder have been formed into particles by extrusion or pelletizing. Still another option can be to use a binder that is a hydrogenation component and/or that includes a hydrogenation component.

After treatment with the hydrogenation component, the catalyst is usually dried by heating at a temperature of 65° C. to 160° C., typically 110° C. to 143° C., for at least 1 minute and generally not longer than 24 hours, at pressures ranging from 100 to 200 kPa-a. Thereafter, the molecular sieve may be calcined in a stream of dry gas, such as air or nitrogen, at temperatures of from 260° C. to 650° C. for 1 to 20 hours. Calcination is typically conducted at pressures ranging from 100 to 300 kPa-a.

In addition, prior to contacting the catalyst composition with the hydrocarbon feed, the hydrogenation component can optionally be sulfided. This is conveniently accomplished by contacting the catalyst with a source of sulfur, such as hydrogen sulfide, at a temperature ranging from about 320 to 480° C. The source of sulfur can be contacted with the catalyst via a carrier gas, such as hydrogen or nitrogen. Sulfiding per se is known and sulfiding of the hydrogenation component can be accomplished without more than routine experimentation by one of ordinary skill in the art in possession of the present disclosure.

In various aspects, a feedstock for transalkylation can include one or more aromatic compounds containing at least 9 carbon atoms. Specific C₉₊ aromatic compounds found in a typical feed include mesitylene (1,3,5-trimethylbenzene), durene (1,2,4,5-tetramethylbenzene), hemimellitene (1,2,4-trimethylbenzene), pseudocumene (1,2,4-trimethylbenzene), 1,2-methylethylbenzene, 1,3-methylethylbenzene, 1,4-methylethylbenzene, propyl-substituted benzenes, butyl-substituted benzenes, and dimethylethylbenzenes. Suitable sources of the C₉₊ aromatics are any C₉₊ fraction from any refinery process that is rich in aromatics. This aromatics fraction contains a substantial proportion of C₉₊ aromatics, e.g., at least 80 wt % C₉₊ aromatics, wherein preferably at least 80 wt %, and more preferably more than 90 wt %, of the hydrocarbons will range from C₉ to C₁₂. Typical refinery fractions which may be useful include catalytic reformate, fluid catalytic cracked (FCC) naphtha or thermal-catalytic cracked (TCC) naphtha.

The feedstock can also include benzene or toluene. In one practical embodiment, the feed to the transalkylation reactor comprises C₉₊ aromatics hydrocarbons and toluene. The feed may also include recycled/unreacted toluene and C₉₊ aromatic feedstock that is obtained by distillation of the effluent product of the transalkylation reaction itself. Typically, toluene constitutes from 0 to 90 wt %, such as from 10 to 70 wt % of the entire feed, whereas the C₉₊ aromatics component constitutes from 10 to 100 wt %, such as from 30 to 85 wt % of the entire feed to the transalkylation reaction. Additionally or alternately, the feed may include benzene. Hydrogen can also be introduced into the transalkylation process.

The feedstock may be characterized by the methyl over single aromatic ring molar ratio. In some embodiments, the combined feedstock (the combination of the C₉₊ and the C₆-C₇ aromatic feedstocks) has a methyl over single aromatic ring molar ratio in the range of from 0.5 to 4, preferably from 1 to 2.5, more preferably from 1.5 to 2.25. The methyl over single aromatic ring molar ratio may be adjusted by adjusting relative flowrate of the C₉₊ and the C₆-C₇ aromatic feedstocks and/or the relative C₆/C₇ ratio of the C₆-C₇ aromatic feedstock.

The transalkylation process can be used to convert a portion of C₉ aromatics and/or C₁₀ aromatics into xylenes. Optionally, the feed can include a reduced or minimized amount of C₉ or C₉₊ aromatics having ethyl or propyl side chains. In some optional aspects, the feedstock can also include at least about 1 wt % of polynuclear aromatics, or at least about 2 wt %, or at least about 5 wt %, or at least about 10 wt %.

In some optional aspects, the feedstock can have a reduced or minimized content of aromatics that are substituted with C₂₊ alkyl side chains. Because xylenes include only methyl (C₁) side chains, any C₂₊ alkyl side chains in the reaction environment can tend to reduce the amount of xylene formation. For liquid phase transalkylation, it can be preferable to use a feedstock where less than about 5 wt % of the aromatics in the feedstock include a C₂₊ side chain, or less than about 2 wt %, or less than about 1 wt %.

Generally, the conditions employed in a liquid phase transalkylation process can include a temperature of about 400° C. or less, or about 360° C. or less, or about 320° C. or less, and/or at least about 100° C., or at least about 200° C., such as between 100° C. to 400° C., or 100° C. to 340° C., or 230° C. to 300° C.; a pressure of 2.0 MPa-g to 10.0 MPa-g, or 3.0 MPa-g to 8.0 MPa-g, or 3.5 MPa-g to 6.0 MPa-g; an H₂:hydrocarbon molar ratio of 0 to 20, or 0.01 to 20, or 0.1 to 10; and a weight hourly space velocity (“WHSV”) for total hydrocarbon feed to the reactor(s) of 0.1 to 100 hr⁻¹, or 1 to 20 hr⁻¹. Optionally, the pressure during transalkylation can be at least 4.0 MPa-g. It is noted that H₂ is not necessarily required during the reaction, so optionally the transalkylation can be performed without introduction of H₂. The feed can be exposed to the transalkylation catalyst under fixed bed conditions, fluidized bed conditions, or other conditions that are suitable when a substantial liquid phase is present in the reaction environment.

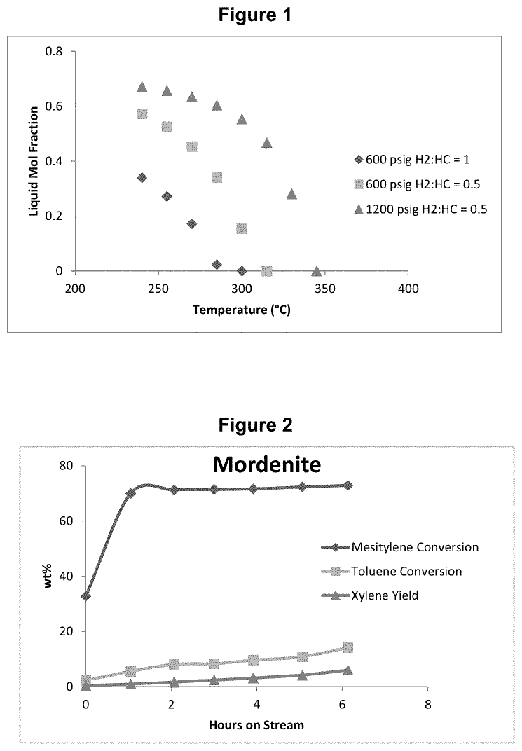

In addition to staying within the general conditions above, the transalkylation conditions can be selected so that a desired amount of the hydrocarbons (reactants and products) in the reactor are in the liquid phase. FIG. 1 shows calculations for the amount of liquid that should be present for a feed corresponding to a 1:1 mixture of toluene and mesitylene at several conditions that are believed to be representative of potential transalkylation conditions. The calculations in FIG. 1 show the mole fraction that is in the liquid phase as a function of temperature. The three separate groups of calculations shown in FIG. 1 correspond to a vessel containing a specified pressure based on introducing specified relative molar volumes of the toluene/mesitylene feed and H₂ into the reactor. One data set corresponds to a 1:1 molar ratio of toluene/mesitylene feed and H₂ at 600 psig (˜4 MPa-g). A second data set corresponds to a 2:1 molar ratio of toluene/mesitylene feed and H₂ at 600 psig (˜4 MPa-g). A third data set corresponds to a 2:1 molar ratio of toluene/mesitylene feed and H₂ at 1200 psig (˜8 MPa-g).

As shown in FIG. 1, temperatures below about 260° C. can lead to formation of a substantial liquid phase (liquid mole fraction of at least 0.1) under all of the calculated conditions, including the combination of the lower pressure (600 psig) and the lower ratio of feed to hydrogen (1:1) shown in FIG. 1. It is noted that based on a ratio of feed to hydrogen of 1:1, a total pressure of 600 psig corresponds to partial pressure of aromatic feed of about 300 psig. Higher temperatures up to about 320° C. can also have a liquid phase (at least 0.01 mole fraction), depending on the pressure and relative amounts of reactants in the environment. More generally, temperatures such as up to 360° C. or up to 400° C. or greater can also be used for liquid phase transalkylation, so long as the combination of temperature and pressure in the reaction environment can result in a liquid mole fraction of at least 0.01. It is noted that conventional transalkylation conditions typically involve temperatures greater than 350° C. and/or pressures below 4 MPag, but such conventional transalkylation conditions do not include a combination of pressure and temperature that results in a liquid mole fraction of at least 0.01.

The resulting effluent from a (liquid phase) transalkylation process can have a xylene yield, relative to the total weight of the hydrocarbons in the effluent, of at least about 4 wt %, or at least about 6 wt %, or at least about 8 wt %, or at least about 10 wt %.

A transalkylation process produces an effluent that can be separated to form one or more output streams based on boiling point or distillation. A first output stream separated from a transalkylation effluent can be a C₆-C₇ stream (possibly including unreacted C₆-C₇ compounds), which can be at least partially recycled to the transalkylation. A second output stream separated from the transalkylation effluent can correspond to C₉₊ compounds (possibly including unreacted C₉₊ compounds). A third output stream separated from the transalkylation effluent can correspond to a C₈ aromatics stream. Optionally, one of the first output stream, second output stream, or third output stream can correspond to a remaining portion of the methylation effluent after separation of other output streams.

In some embodiments, naphthalene-containing streams, such as those found in various refinery and/or chemical plant streams may be processed through a liquid phase transalkylation process to form useful products and/or streams. For example, in some embodiments, the feedstock for such a liquid phase transalkylation process may include 8-24 wt % naphthalene and 60-90 wt % C₉ alkylbenzenes. In one specific embodiment, the feedstock includes a combination of a first feedstock stream and a second feedstock stream. The first feedstock stream comprises about 2.6 wt % trimethylbenzene, about 1.3wt % indane, about 1.9 wt % diethylbenzene, 7.8 wt % methylpropylbenzene, about 34.1 wt % dimethylethylbenzene, about 13.8 wt % tetramethylbenzene, about 9.6 wt % methylindanes, about 8.3 wt % naphthalene, and about 20 wt % other components (e.g., heavier compounds). The second feedstock stream comprises about 59.1 wt % methylethylbenzene, about 24.0 wt % naphthalene, about 6.2 wt % methylnaphthalene, and about 10.7 wt % other components (e.g., heavier compounds). The first feedstock stream and the second feedstock stream may be taken from any suitable source, such as product, effluent, or other streams found at refineries and/or chemical plants. In addition in other embodiments the feed may be derived from coal, fluid catalytic cracking (FCC), light cycle oil (LCO), C1R, methane reforming, C2A, ethane reforming, or combinations thereof.

The first and second feedstock streams described above may be blended in any suitable ratio prior to routing the blended feedstock (i.e., a blended feedstock comprising some percentage of the first feedstock stream and the second feedstock stream) to the transalkylation catalyst in the liquid phase. In some embodiments, the molar ratio of the first feedstock stream to the second feedstock stream may be approximately 1:1. In at least some embodiments, the transalkylation feedstock (e.g., the first feedstock stream, the second feedstock stream, some combination of the first feedstock stream and second feedstock stream, or some other feedstock stream) has a naphthalene concentration of 5 to 50 wt % relative to the total amount of the feedstock.

In at least some embodiments, the mole fraction of the feedstock (i.e., the first feedstock stream and/or the second feedstock stream) in the liquid phase relative to the total amount of the feedstock is at least 0.01, or at least 0.05, or at least 0.08, or at least 0.1, or at least 0.15, or at least 0.2, or at least 0.3, or at least 0.4, or at least 0.5, and optionally up to having substantially all of the feedstock in the liquid phase.

In some embodiments, the transalkylation catalyst may comprise MCM-49, USY, Beta, or some combination thereof. In addition, the reaction conditions for the liquid phase transalkylation may comprise a temperature between 200° C. and 600° C., a pressure between 100 and 800 psig, and a weight hourly space velocity (“WHSV”) for total hydrocarbon feed to the reactor(s) of 0.5 to 5.0 hr⁻¹.

The resulting transalkylated product from the above described liquid phase transalkylation process may comprise 99.0% or more aromatic compounds, and may have a mixed aniline point (which is a measure of solvency) of less than 15° C. In some embodiments, the transalkylated product resulting from the above described liquid phase transalkylation process may be suitable for use in or as, among other things, a heavy aromatics solvent (e.g., such as those utilized in agricultural applications) and compressor wash oil.

### EXAMPLES 1-6

### Liquid Phase Transalkylation with Various Catalysts

In Examples 1-6, several types of catalysts having 12-member ring channels and/or active sites were used for transalkylation reactions. The catalysts were exposed to a feed composed of chemical grade toluene and mesitylene in 1:1 molar ratio (57:43 weight ratio) at an initial temperature of 220° C., a 1:1 molar ratio of feed to H₂, and a weight hourly space velocity of about 6 hr⁻¹. The reaction was conducted with an up-flow fixed-bed unit equipped with a 5-mm (inside-diameter) stainless steel reactor. Once the reaction reached steady-state, the product stream was analyzed with an online GC. Product analysis was repeated while the reactor temperature was increased at 10° C. intervals from 220 to 400° C. The results obtained at 260° C. are shown in Table 1.

For Example 1, a ZSM-12 catalyst was used. The catalyst was bound with 35% alumina to 1/16″ cylindrical extrudate, steamed at 900° F. for 5.25 hours, and cut to 1/16″ length. The catalyst was then dried with flowing N₂ at 250° C. and 300 psig (2.1 MPag) for 3 hours prior to exposure to the feed. It is noted that the catalyst does not include a separate hydrogenation metal. The catalysts for Examples 2-6 were prepared in a similar manner with respect to catalyst weight, binder amount, catalyst size, and other steaming/drying conditions. The catalyst for Example 2 included zeolite Beta instead of ZSM-12. Example 3 included ultrastable Y. Example 4 included mordenite. Example 5 included MCM-49. Example 6 included MCM-56. For each catalyst, the ratio of Si/Al₂ in the catalyst is also shown in Table 1.

The transalkylation conditions shown in Table 1 correspond to about 300 psig (˜2.1 MPa-g) and ˜260° C. Under these conditions, the mole fraction of the hydrocarbon feed corresponding to a liquid is believed to be between 0.01 and 0.1. Therefore, the conditions in Table 1 are believed to correspond to having a small but distinct amount of liquid phase feed (and optionally products) in the reaction environment.

TABLE 1
Catalyst and Product Analysis for Examples 1-6

| | | | | | p- | | | | |
|---|---|---|---|---|---|---|---|---|---|
| | Catalyst | | | | xylene / | Mesitylene/ | Mesitylene | Xylene/ | C10 |
| | (Si/Al₂ | Toluene | Mesitylene | Xylene | total | total | approach | Bz | Arom. |
| | ratio of | Conv | Conv | Yield | xylene | TMB | equilibrium | molar | Yield |
| Ex. | zeolite) | % | % | % | % | % | % | ratio | % |
| 1 | ZSM-12 | 5 | 52 | 1 | 27 | 44 | 75 | 10 | 1 |
| | (200:1) | | | | | | | | |
| 2 | Beta | 14 | 70 | 12 | 28 | 34 | 90 | 28 | 4 |
| | (38:1) | | | | | | | | |
| 3 | USY | 10 | 23 | 6 | 25 | 76 | 22 | 27 | 2 |
| | (10:1) | | | | | | | | |
| 4 | Mordenite | 3 | 66 | 4 | 25 | 33 | 92 | 14 | 1 |
| | (19:1) | | | | | | | | |
| 5 | MCM-49 | 7 | 52 | 0.5 | 28 | 46 | 70 | 2 | 2 |
| | (18:1) | | | | | | | | |
| 6 | MCM-56 | 4 | 47 | 0.1 | 29 | 50 | 64 | 3 | 1 |
| | (18:1) | | | | | | | | |

The ZSM-12 catalyst in Example 1 corresponds to a 1-dimensional 12-member ring molecular sieve that has previously been used for gas phase transalkylation. This is described, for example, in the Examples provided in U.S. Pat. No. 8,163,966.

As shown in Table 1, ZSM-12 provides only a minimal xylene yield when transalkylation is performed at a temperature that is sufficiently low to allow a substantial liquid phase to be present. Additionally, this low or minimal xylene yield is produced after approaching ˜75% of the way to equilibrium with regard to mesitylene conversion. This indicates that attempting to perform liquid phase transalkylation with ZSM-12 has a low selectivity for xylene formation. This is in contrast to the xylene yield for the molecular sieves in Examples 2 and 3 (Beta and USY), which include 3-dimensional 12-member ring pore networks. Without being bound by any particular theory, it is believed that the 3-dimensional 12-member ring pore networks can provide additional activity advantages under liquid phase transalkylation conditions. Even though only a modest amount of liquid phase feed is present at 260° C. and 300 psig, the 3-dimensional 12-member ring molecular sieves of Examples 2 and 3 provide substantial selectivity for xylene formation relative to ZSM-12.

With regard to Example 4, mordenite corresponds to a 1-dimensional 12-member ring molecular sieve with a larger pore channel size than ZSM-12. Mordenite appears to provide improved selectivity for xylene formation relative to ZSM-12. This suggests that other large pore diameter 1-dimensional 12-member ring zeolites may also be able to provide improved selectivity. ZSM-18 is another example of a 1-dimensional 12-member ring zeolite with a larger pore channel size than ZSM-12.

Examples 5 and 6 show results from exposing the toluene/mesitylene feed to MWW framework catalysts. Both MCM-49 (Example 5) and MCM-56 (Example 6) appear to show minimal selectivity for xylene formation under transalkylation conditions when used without an additional hydrogenating metal.

The amount of mesitylene conversion is also an indicator for transalkylation activity. Table 1 shows that the zeolite Beta catalyst (Example 2) has the highest conversion of mesitylene (about 70%). Although most of the mesitylene is converted to other trimethylbenzenes, this still demonstrates a high general activity for transalkylation. It is noted that mordenite (Example 4) had a similar mesitylene conversion with a lower xylene yield.

The USY catalyst (Example 3) appears to have a low mesitylene conversion of about 23%, even though the USY catalyst also provided the second highest xylene yield. This suggests a potential for increased xylene yields using a USY (or other FAU framework molecular sieve) under conditions tailored for use with the catalyst.

### EXAMPLES 7-10

### Batch Transalkylation

In separate batch experiments using the same 1:1 mesitylene/toluene feed (mole/mole) in liquid phase at ˜260° C. and about 400 psig (˜2.8 MPa-g), several catalysts were evaluated for xylene production. For the batch runs in Examples 7-10, fifteen grams of catalyst were dried at 260° C. for one hour and then loaded into a catalyst basket. For the MCM-22 catalyst in Example 8, the catalyst was unbound, and only 13.5 grams of the MCM-22 catalyst were loaded into the catalyst basket. For each of Examples 7-10, the catalyst basket and 150 g feed were placed inside a 600-mL autoclave reactor. The reactor temperature was ramped to 260° C. and then pressurized with H₂ while maintaining a 500 rpm stirring rate until a pressure of 400 psig was achieved. Once the temperature reached 260° C., a 2-cc sample was collected for GC analysis. Sample collection continued at 1-hour intervals for 5 hours. The reactor was cooled down afterwards. The catalyst for Example 7 was mordenite (65 wt % binder/35 wt % alumina binder); for Example 8 was MCM-22 (80/20 alumina bound); for Example 9 was FAU (80/20 alumina bound) that also included 0.15 wt % of Pd supported on the catalyst; and for Example 10 was MCM-49 (80/20 alumina bound). The results from Examples 7-10 are shown in FIGS. 2-5.

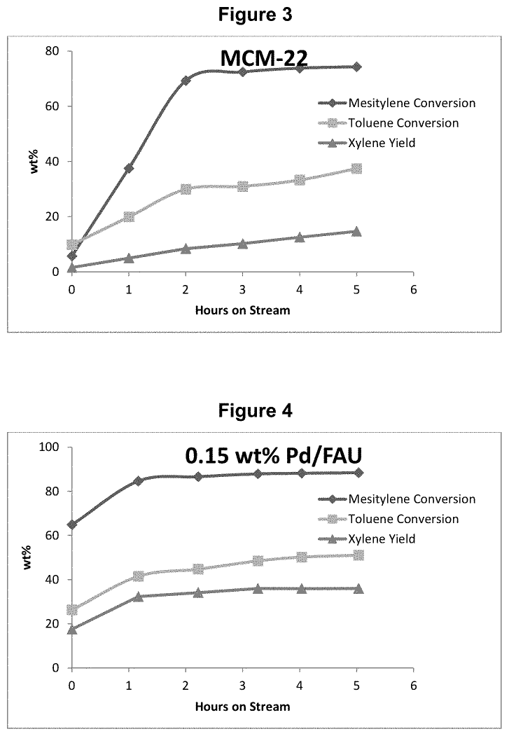

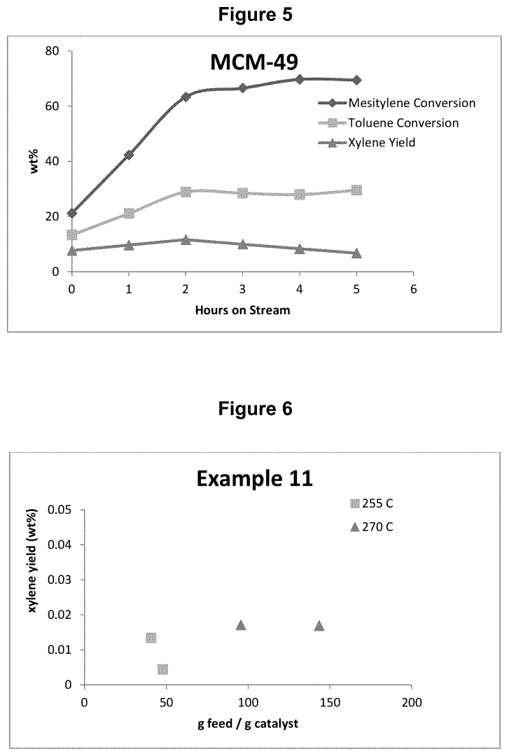

FIG. 2 (Example 7) shows that mordenite has activity for mesitylene conversion under the batch conditions which include a liquid phase, but with low or reduced amounts of toluene conversion and xylene production. MCM-22 (FIG. 3, Example 8) and MCM-49 (FIG. 5, Example 10) both demonstrate somewhat higher amounts of toluene conversion and xylene production. The addition of 0.15 wt % Pd to FAU (FIG. 4, Example 9) provided a catalyst that also appeared to provide a high activity for formation of xylene. The amount of mesitylene and toluene converted in FIG. 4 is believed to correspond to equilibrium amounts of conversion.

### EXAMPLES 11-16

### Additional Characterization of Transalkylation Products

In Examples 11-16, a 1:1 molar ratio toluene/mesitylene feed was exposed to various catalysts at a total pressure of about 600 psig (˜4.1 MPa-g) and temperatures ranging from 240° C. to 380° C. Processing was started at 240° C., and then increased by 15° C. intervals to 300° C. to investigate various processing conditions. Runs were also performed for some catalysts at 325° C. and 380° C. The feed was introduced into the reactor at a weight hourly space velocity of ˜2 hr⁻¹. The molar ratio of hydrogen to feed was about 1:1. The catalysts used for transalkylation were MCM-49 (Example 11); MCM-49 with 0.15 wt % Pd (Example 12); zeolite Beta with a Si/Al₂ ratio of about 40 (Example 13); zeolite Beta (Si/Al₂) ratio about 40) with 0.15 wt % Pd (Example 14); a second version of zeolite Beta with a ratio of Si to Al₂ of about 200 (Example 15); and FAU (Example 16). All of the catalysts were bound with alumina in a 80:20 weight ratio.

FIG. 6 shows xylene yield for Example 11, corresponding to processing in the presence of MCM-49. The catalyst has low activity at the temperatures shown, resulting in limited production of xylene.

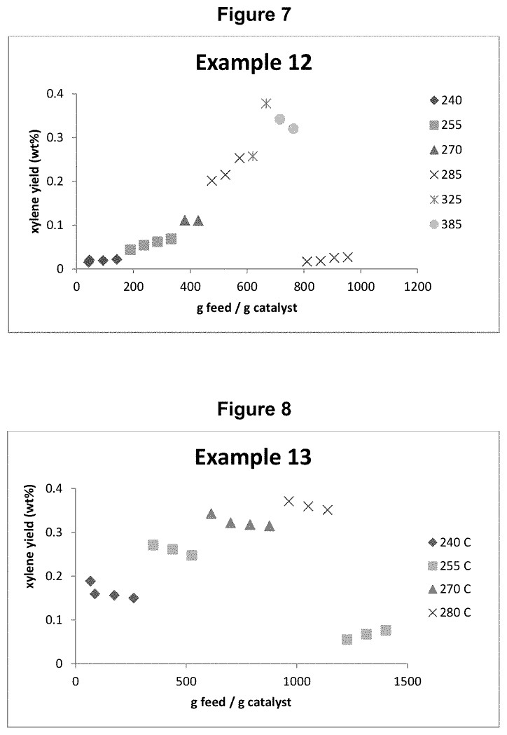

FIG. 7 shows xylene yield for Example 12, corresponding to processing in the presence of MCM-49 with 0.15 wt % of Pd supported on the catalyst. The addition of 0.15 wt % Pd appears to increase the activity of the MCM-49 for converting the feed to xylene. It is noted that a 20+ wt % xylene yield on feed, which occurred at temperatures of 285° C. and greater, is a yield that might be found in a commercial gas phase process. However, such yields in a gas phase process can typically require a transalkylation temperature of greater than 380° C. It is noted that the final data points corresponding to 285° C. represent using the same catalyst for a final processing run. The lower activity for the final run at 285° C. indicates that some catalyst deactivation may be occurring over time. However, see Example 17 below for evidence that processing at approximately constant temperature may not result in substantial catalyst deactivation over extended periods.

FIG. 16 shows a further comparison of the activity of MCM-49 with and without a supported hydrogenation metal under another set of transalkylation conditions. For the processes in FIG. 16, either an MCM-49 catalyst or a 0.15 wt % Pd/MCM-49 catalyst was exposed to a feed with a 1:1 ratio of mesitylene to toluene at a total pressure of about 300 psig (˜2.1 MPa-g) and a range of temperatures starting at about 280° C.

FIG. 8 shows xylene yield for Example 13, corresponding to processing in the presence of zeolite Beta with a Si/Al₂ ratio of about 38. The zeolite Beta results in substantial production of xylene, even though all of the reaction temperatures shown are below 300° C. Again, a final portion of the run at lower temperature may indicate some catalyst deactivation over time after performing transalkylation at a higher temperature.

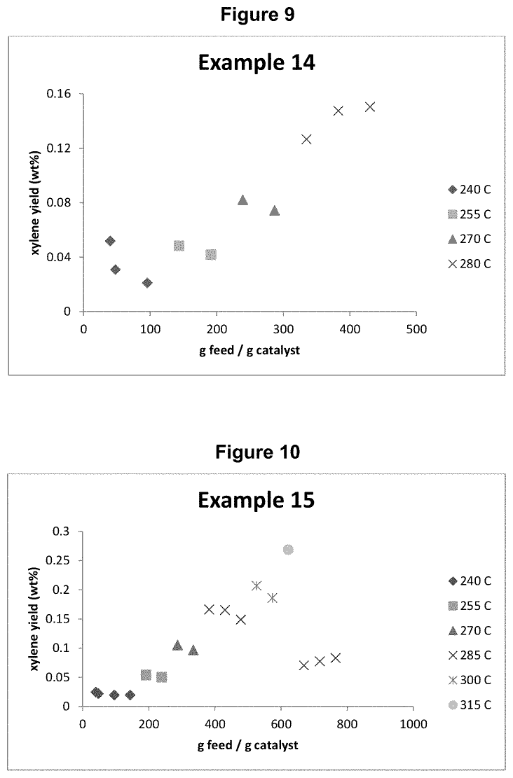

FIG. 9 shows xylene yield for Example 14, corresponding to processing in the presence of zeolite Beta (Si/Al₂ ratio of about 38) with 0.15 wt % of Pd on the catalyst. Addition of a hydrogenation metal to zeolite Beta appears to significantly reduce xylene production. This is in contrast to the results shown in Example 12/FIG. 7, where addition of a hydrogenation metal to an MWW framework catalyst resulted in an improvement in activity for xylene production.

FIG. 10 shows xylene yield for Example 15, corresponding to processing in the presence of zeolite Beta with a higher Si/Al₂ ratio of approximately 200. The activity for xylene production of the higher Si/Al₂ ratio Beta is lower than the Beta from Example 13.

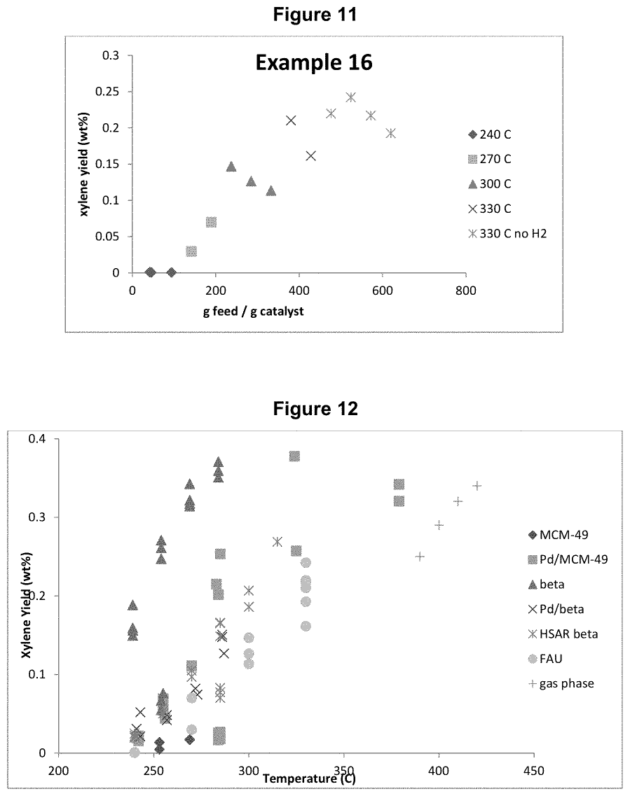

FIG. 11 shows xylene yield for Example 16, corresponding to processing in the presence of an FAU catalyst. FIG. 11 appears to show that FAU has some activity for xylene production. Additionally, when H₂ was removed from the reaction environment at a temperature of about 330° C., an increase in activity of about 50% was observed relative to the transalkylation in the presence of hydrogen at 330° C.

FIG. 12 combines the xylene yield results from Examples 11-16 into a single plot. FIG. 12 also shows xylene production under gas phase conditions using a catalyst system corresponding to a mix of ZSM-5 and ZSM-12. The feed for the gas phase ZSM-5/ZSM-12 system was a mixture of aromatics. The aromatic mixture was processed at 350 psig, a weight hourly space velocity of 6 hr⁻¹, and a temperature of 380° C. to 440° C. FIG. 12 shows that zeolite Beta (both ratios) and the MCM-49 with 0.15 wt % Pd show a substantial activity advantage in terms of temperature to achieve a specified yield relative to processing with the ZSM-5/ZSM-12 catalyst system under gas phase conditions. For example, zeolite Beta (Si/Al₂ ratio of 38) has an activity advantage of about 140° C., while the higher Si/Al₂ ratio Beta has an activity advantage of about 60° C.

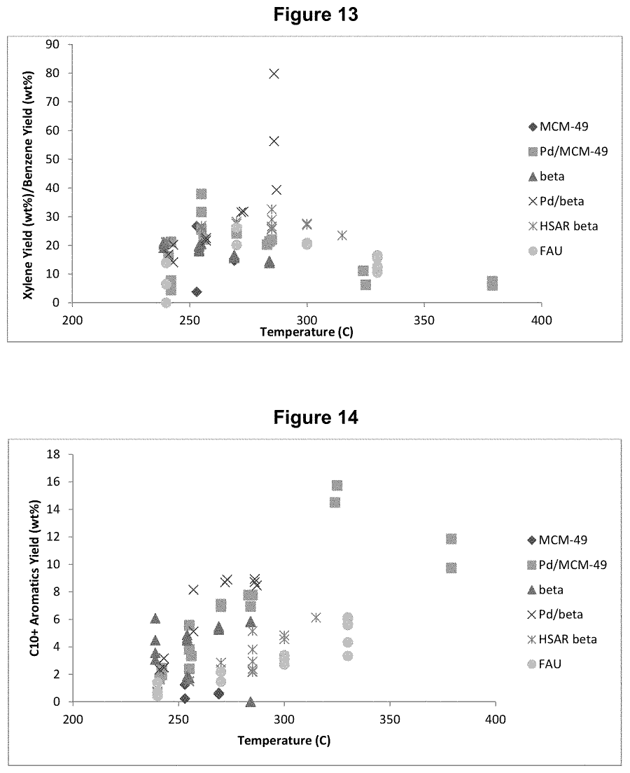

FIG. 13 shows the relative amounts of xylene yield and benzene yield during the processing runs of Examples 11-16. Generally, the benzene yield from all of the processing runs of Examples 11-16 was less than 1 wt %. As a result, most of the variation in xylene to benzene yield ratio is due to differences in xylene yield between the catalysts.

FIG. 14 shows the amount of production of heavy aromatic compounds (C₁₀₊) during the processing runs of Examples 11-16. FIG. 14 shows that catalysts that included Pd as a supported hydrogenation metal resulted in increased formation of heavy aromatic compounds.

### EXAMPLE 17

### Catalyst Stability

As a test of catalyst stability, naphthalene conversion under transalkylation conditions was performed on feeds containing mixtures of aromatics. Although naphthalene is a 2-ring aromatic, it is believed that the stability during naphthalene conversion can be similar to the stability during transalkylation of 1-ring aromatics. Also, during gas phase transalkylation, the presence of C₁₀₊ aromatics is believed to result in catalyst deactivation. Thus, stability in the presence of C₁₀₊ compounds can be valuable for a transalkylation catalyst.

Transalkylation of naphthalene was performed using two types of feedstocks. One feedstock corresponded to Feed A in Table 2. The second feedstock corresponded to a 2:1 (by weight) blend of Feed B and Feed A.

TABLE 2
Feeds for Naphthalene Transalkylation

| Component, Wt % | Feed A | Feed B |
|---|---|---|
| Trimethylbenzenes | 2.6 | 0.0 |
| Methylethylbenzenes | 0.0 | 59.1 |
| Indane | 1.3 | 0.0 |
| Diethylbenzenes | 1.9 | 0.0 |
| Methylpropylbenzenes | 7.8 | 0.0 |
| Dimethylethylbenzenes | 34.1 | 0.0 |
| Tetramethylbenzenes | 13.8 | 0.0 |
| Methylindanes | 9.6 | 0.0 |
| Naphthalene | 8.3 | 24.0 |
| Methylnaphthalene | 0.0 | 6.2 |
| Bottoms | 20.6 | 10.7 |

As can be seen from Table 2, Feed A is a complex mixture of C₉-C₁₀ with the majority being C₁₀ aromatics. Dimethylethylbenzene is one of the key components in Feed A. Feed A also contains about 8% naphthalene. Feed B is composed of 59% methylethylbenzenes with 24% naphthalene and 6% methylnaphthalene. It could be representative, for example, of the type of fraction that might be generated as a remaining or bottoms fraction from a process for methylating toluene to form xylenes. In the second feedstock, Feed B was tested in 2/1 admixture with Feed A. The “bottoms” portion of both Feed A and Feed B represents heavier components and/or components with more than 2 aromatic rings.

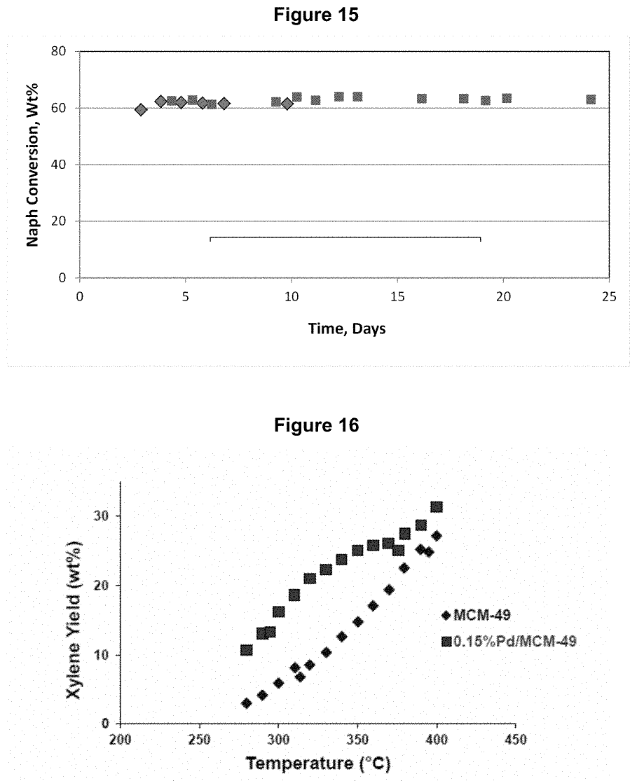

The feedstocks were exposed to an MCM-49 catalyst at 275° C., a weight hourly space velocity of 1.5 hr⁻¹, and a pressure of about 500 psig over a period of days. FIG. 15 shows the amount of naphthalene conversion as measured over time during the transalkylation runs. As shown in FIG. 15, the naphthalene conversion was stable during exposure of both types of feedstocks to the catalyst. For the second feedstock, the transalkylation activity remained stable over the course of 24 days of processing.

### EXAMPLE 18

### Liquid Phase Transalkylation of Napthalene-Containing Streams

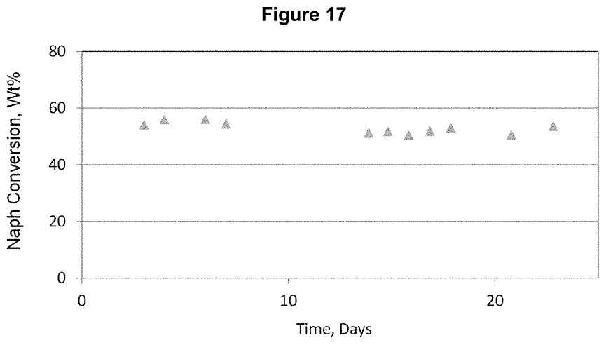

In Example 18, a 1:1 weight ratio blend of Feeds A and B from Table 2 above were exposed to an MCM-49 catalyst at a temperature of 280° C., a weight hourly space velocity of 1.5 hr⁻¹, and a pressure of about 500 psig over a period of days. FIG. 17 shows the amount of naphthalene conversion as measured over time during the transalkylation run. As shown in FIG. 17, the naphthalene conversion was substantially stable during the twenty-five day exposure, with an average conversion rate of about 54%. The resulting transalkylate product was distilled, and the 235-305° C. fraction was found to have an aromaticity of 99%+, and a mixed anline point of about 13° C.

While the disclosed embodiments have been described and illustrated with respect to certain aspects, it is to be understood that the invention is not limited to the particulars disclosed and extends to all equivalents within the scope of the claims. Unless otherwise stated, all percentages, parts, ratios, etc. are by weight. Unless otherwise stated, a reference to a compound or component includes the compound or component by itself as well as in combination with other elements, compounds, or components, such as mixtures of compounds. Further, when an amount, concentration, or other value or parameter is given as a list of upper preferable values and lower preferable values, this is to be understood as specifically disclosing all ranges formed form any pair of an upper preferred value and a lower preferred value, regardless of whether ranges are separately disclosed. All patents, test procedures, and other documents cited herein, including priority documents, are fully incorporated by reference to the extent such disclosure is not inconsistent and for all jurisdictions in which such incorporation is permitted.

1. A method for liquid phase transalkylation of aromatic compounds, comprising:
exposing an aromatic feedstock comprising C₉₊ aromatics and at least one of benzene and toluene to a transalkylation catalyst under effective transalkylation conditions to form a transalkylation effluent;
wherein the mole fraction of aromatic compounds in the liquid phase in the feedstock, relative to the total amount of aromatic compounds in the feedstock, is at least about 0.01 under the effective transalkylation conditions;
wherein the transalkylation effluent has a higher weight percentage of C₈ aromatics than the feedstock; and
wherein the catalyst comprises at least one of the following:
a molecular sieve with a 3-dimensional 12-member ring or larger pore network;
a molecular sieve with a 1-dimensional 12-member ring or larger pore network, wherein the 1-dimensional channel has a pore channel size of at least 6.0 Angstroms;
an acidic microporous material with a pore channel size of at least 6.0 Angstroms; and
a molecular sieve having a MWW framework.

2. The method of claim 1, wherein the molecular sieve comprises a 3-dimensional 12-member ring or larger pore network.

3. The method of claim 2, wherein the molecular sieve comprises a framework structure selected from the group consisting of FAU, CON, EMT, MSE, ISV, IWR, IWV, a Beta polymorph, or a combination thereof.

4. The method of claim 1, wherein the molecular sieve has a framework structure selected from the group consisting of FAU, EMT, a Beta polymorph, or a combination thereof.

5. The method of claim 4, wherein the molecular sieve has a Beta polymorph framework structure and a Si/Al₂ ratio of about 10 to about 400.

6. The method of claim 4, wherein the molecular sieve has a FAU framework structure and a Si/Al2 ratio of about 2 to about 400.

7. The method of claim 1, wherein the molecular sieve has a 1-dimensional 12-member ring or larger pore network.

8. The method of claim 7, wherein the molecular sieve has a framework structure of MOR, MEI, or a combination thereof.

9. The method of claim 1, wherein the acidic microporous material or the molecular sieve has a pore channel size of at least 6.3 Angstroms.

10. The method of claim 1, wherein the molecular sieve comprises MCM-22, MCM-49, MCM-56, or a combination thereof.

11. The method of claim 1, wherein the mole fraction of aromatic compounds in the liquid phase in the feedstock, relative to the total amount of aromatic compounds in the feedstock, is at least about 0.1 under the effective transalkylation conditions.

12. The method of claim 1, wherein the effective transalkylation conditions comprise a temperature of less than 280° C., a total pressure of at least 4.0 MPag, a molar ratio of H₂ to hydrocarbons in the feedstock of about 0.01 to 20, or a combination thereof.

13. The method of claim 1, wherein the catalyst further comprises 0.01 wt % to 5 wt % of at least one metal from Groups 5-11 and 14.

14. The method of claim 13, wherein the at least one metal from Groups 5-11 and 14 is selected from the group consisting of Pd, Pt, Ni, Rh, Cu, Sn, Fe, W, V, Mo, Re, Cr, Mn, Ru, Os, Co, Ir, or a combination thereof.

15. The method of claim 14, wherein the at least one metal from Group 5-11 and 14 is Pd, Pt, Ni, or a combination thereof.

16. The method of claim 13, wherein the catalyst comprises a bimetallic metal.

17. The method of claim 16, wherein the bimetallic metal is Pt/Sn, Pt/Cu, Pt/Pd, Pt/Rh, or a combination thereof.

18. The method of claim 1, wherein the feedstock further comprises at least 2 wt % naphthalene, at least 1 wt % polynuclear aromatics, less than 5 wt % of the aromatics in the feedstock comprise a C₂₊ side chain, or a combination thereof.

19. A method for liquid phase transalkylation of aromatic compounds, comprising:
exposing an aromatic feedstock comprising C₉₊ aromatics and at least one of benzene and toluene to a transalkylation catalyst under effective transalkylation conditions to form a transalkylation effluent;
wherein the effective transalkylation conditions comprise a temperature of less than 280° C., a total pressure of at least 4.0 MPag, a molar ratio of H₂ to hydrocarbons in the feedstock of about 0.01 to 20, or a combination thereof;
wherein the mole fraction of aromatic compounds in the liquid phase in the feedstock, relative to the total amount of aromatic compounds in the feedstock, is at least about 0.1 under the effective transalkylation conditions;
wherein the transalkylation effluent has a higher weight percentage of C₈ aromatics than the feedstock; and
wherein the catalyst comprises a molecular sieve having an MWW framework and 0.01 wt % to 5.0 wt % of Pd;
wherein the molecular sieve comprises MCM-22, MCM-49, MCM-56, or a combination thereof.

20. The method of claim 19, wherein the feedstock further comprises at least 2 wt % naphthalene, at least 1 wt % polynuclear aromatics, less than 5 wt % of the aromatics in the feedstock comprise a C₂₊ side chain, or a combination thereof.

21. A method for liquid phase transalkylation, the method comprising:
exposing a feedstock comprising 5 to 50 wt % naphthalene and one or more alkylbenzenes to a transalkylation catalyst to form a transalkylation effluent;
wherein the mole fraction of the feedstock in the liquid phase relative to the total amount of the feedstock is at least about 0.1 under effective transalkylation conditions; and
wherein the transalkylation effluent comprises 99.0% or more aromatic compounds and an aniline point of 15° C. or less.

22. The method of claim 21, wherein the transalkylation catalyst comprises MCM-49.

23. The method of claim 21, wherein the exposing further comprises exposing the feedstock to the transalkylation catalyst at a temperature of approximately 200° C. to 600° C., a weight hourly space velocity of approximately 0.5 to 5.0 hr⁻¹, and a pressure of approximately 100 psig to 800 psig.
